(19) United States
(12) Patent Application Publication
(10) Pub. No.: US 2003/0060413 A1
(43) Pub. Date: Mar. 27, 2003

(54) Derivatives of pseudo-peptides, their preparation and their biological uses

(76) Inventor: Robert H. Zimmer, Mulhouse (FR)

Correspondence Address: Cummings & Lockwood, 700 State Street, P.O. Box 1960, New Haven, CT, 06509-1960, US

(21) Appl. No.: 10/237,254

(22) Filed: Sep. 6, 2002

Related U.S. Application Data
(60) Provisional application No. 60/317,736, filed on Sep. 6, 2001.

(51) Int. Cl. A61K 38/16; C07K 14/16; C07K 7/08

(52) U.S. Cl. 514/12; 530/324

(57) ABSTRACT

Disclosed herein is a prodrug for use in the treatment of physiological conditions comprising a carrier moiety selected from the group consisting essentially of cinnamoyl, benzoyl, phenylacetyl, 3,4-methylenedioxycinnamoyl and 3,4,5-trimethoxycinnamoyl, wherein the carrier moiety is chemically linked to a therapeutic pseudo-polypeptide of the formula aaₙ, where aa is a chemically modified amino acid, or a chemical or structural variation thereof, where n is an integer from 2 to 40, and wherein the pseudo-polypeptide is poorly absorbed orally.

In an alternative variation, the prodrug of the present invention further comprises a non-therapeutic linker species linking the pseudo-polypeptide to the carrier moiety. Preferably, the linker species is an amino acid. Thus, the prodrug of the present invention can be viewed as a three-component entity: the first, therapeutically active component is the pseudo-polypeptide; the second is the linker species, possibly an additional, non-therapeutic amino acid; and the third is the carrier moiety.

Also disclosed are methods for the enhancement of the bioavailability of orally administered polypeptide substances.

### CROSS-REFERENCE TO RELATED APPLICATIONS

The present application claims priority to provisional patent application serial No. 60/317,737, filed Sep. 6, 2001, the disclosure of which is hereby incorporated specifically by reference.

### FIELD OF THE INVENTION

This invention, in general, relates to novel derivatives of pseudo-peptides and, more particularly, relates to therapeutic pseudo-polypeptides chemically linked to carrier moieties, wherein the carrier moieties are selected from the group including cinnamoyl, benzoyl, phenylacetyl, 3,4 methylenedioxycinnamoyl and 3,4,5-trimethoxycinnamoyl; their preparation; and their biological uses. The invention further relates to prodrugs for the oral administration of therapeutically active pseudo-polypeptides that are otherwise poorly orally absorbable.

### BACKGROUND OF THE INVENTION

It has been observed that therapeutically effective polypeptides and pseudo-polypeptides (aaₙ) having two or more amino acids (n≧2) are poorly absorbed orally.

It has also been observed that pseudo-peptides have a longer in vivo half-life and exhibit an improved stability as compared to the corresponding natural peptides. Furthermore, they are less sensitive to enzymatic degradation and, in certain cases, they exhibit improved biological activities. Lipopeptides have been described as improving the immune response compared to the natural peptide in vivo, most probably by improving the presentation of the antigen and/or by playing the role of an adjuvant. However, the manufacture of such lipopeptides on an industrial scale is very difficult. It requires certain procedures, such as chemical ligations, that depend on the specific amino acid sequences involved. Until the present invention, procedures for improving the biological properties, particularly the immunological properties, of a pseudo-peptide by the coupling of a lipid moiety have not been disclosed.

It would be desirable to provide a cellular immune response in immunizing against agents such as viruses for which antibodies have been shown to enhance infectivity. It would also be useful to provide such a response against both chronic and latent viral infections and against malignant cells.

The “peptide” vaccine concept is based on the identification and chemical synthesis of B cell and T cell epitopes, particularly those that may be immunodominant and induce specific immune functions (neutralization, killing, helping). In their simplest form, peptides used as vaccine candidates are linear polymers of ˜8-24 amino acids. However, in an attempt to mimic conformational structures (particularly those of the viral envelope) cyclic peptides, branched peptides, peptomers (cross-linked peptide polymers) and other complex multimeric structures, as well as peptides conjugated to other molecules have been developed.

The use of synthetic peptide vaccines, although known, does not solve the problems discussed above because either the peptides do not readily associate with histocompatibility molecules, have a short serum half-life, are rapidly proteolyzed, or do not specifically localize to antigen-presenting monocytes and macrophages. At best, all exogenously administered antigens must compete with the universe of self-proteins for binding to antigen-presenting macrophages.

Several obstacles limit the usefulness of peptide vaccines, particularly for the treatment of AIDS: the low immunogenicity of peptide vaccines, the extensive genetic variability of HIV, the dependence of T cell epitopes on the individual's immunogenetic background, the difficulties in translating results from experimental animals (e.g., peptides recognized by mice, rabbits and macaques may be different from those immunogenic in humans); and the lack of knowledge on the structural conformation of the most relevant neutralization epitopes in HIV.

Major efforts have been mounted to elicit immune responses to poorly immunogenic viral proteins from the herpes viruses, non-A, non-B hepatitis, HIV, and the like. These pathogens are difficult and hazardous to propagate in vitro. As mentioned above, synthetic peptide vaccines corresponding to viral-encoded proteins have been made, but have severe limitations. Attempts have also been made to use vaccinia virus vectors to express proteins from other viruses. However, the results have been disappointing, because (a) recombinant vaccinia viruses may be rapidly eliminated from the circulation in already immune individuals; and (b) the administration of complex viral antigens may induce a phenomenon known as “antigenic competition,” in which weakly immunogenic portions of the virus fail to elicit an immune response because they are out-competed by other, more potent regions of the administered antigen.

Another major problem with protein or peptide vaccines is an anaphylactic reaction that can occur when injections of antigen are repeated in efforts to produce a more potent immune response. In this phenomenon, IgE antibodies formed in response to the antigen cause severe and sometimes fatal allergic reactions.

Conjugation of peptides to larger molecules can result in enhanced immunogenicity, particularly if the carrier proteins contain strong T helper epitopes. Direct covalent fusion or cross-linking with glutaraldehyde and other chemical processes have been used. Tetanus toxoid, Pseudomonas aeruginosa toxin A, beta-galactosidase, Brucella abortus (killed bacteria), keyhole limpet hemocyanin, influenza virus hemagglutinin and nucleoprotein, hepatitis B core and surface antigens, etc., are examples of carrier proteins that have successfully improved the immunogenicity of peptides. Some of them (e.g., brucella) appear to induce T-cell independent responses. However most of them act by providing T cell help or facilitating their presentation, functions that require their covalent fusion with the peptides. Experimental attempts to further enhance the immunogenicity of synthetic peptides include fusion with plasma alpha-2 macroglobulin (which has specific receptors in macrophages and may enhance antigen presentation), beta-2 microglobulin, light and heavy immunoglobulin chains, etc. See, for example, Ahlers, et. al., Proc. Natl Sci USA, 1997 94(20):10856-61.

Covalent fusion of peptides to lipids has resulted in conjugates of enhanced immunogenicity, particularly in more efficient induction of CTL's, bypassing the need for other adjuvants. Encouraging results have been reported for hepatitis B core antigen lipopeptide-based vaccine linked to a tetanus toxoid helper T epitope: the magnitude and persistence of the CTL response induced were comparable to those observed in patients who successfully cleared the infection. In the SIV model a Nef-based lipopeptide was shown to induce strong specific CTLs to a Nef determinant in macaques; however, after challenge with the SIV source of the Nef, escape from this epitope occurred, highlighting the need for broad, multi-epitope responses. See, for example, Greenstein, et al., J. Immunol. 1992 148(12):3970-77).

Mucosally-administered peptides have been immunogenic in mice (i.e.,induction of CTL, Th, and antibody), particularly when administered with cholera toxin as adjuvant. In a recent mouse model of rectal immunization/rectal challenge, a multideterminant peptide-based vaccine (containing HIV envelope B and T cell epitopes) was able to induce resistance to a vaccinia recombinant expressing HIV gp160. Protection was dependent on the mucosal presence of CTL's, and was enhanced by co-administration of IL-12 at the time of vaccination. Intestinal induction of HIV neutralizing antibodies has been achieved by oral administration of a multicomponent peptide vaccine when administered with cholera toxin; similarly, CTL's have been induced by mucosal co-administration of a CTL epitope-based peptide and cholera toxin. See, for example, Belyakov, et al., J. Clin. Invest. 1998, 102(12): 2072-81; Belyakov, et al., Proc. Natl Acad. Sci. USA, 1998, 95(4):1709-14.)

Accordingly, there is a need for a method for invoking a safe and effective immune response to this type of protein or polypeptide, more particularly pseudo-polypeptides. Moreover, there is a great need for a method that will associate these antigens with Class I histocompatibility antigens on the cell surface to elicit a cytotoxic T-cell response, avoid anaphylaxis, minimize proteolysis of the material in the serum, and facilitate localization of the material to monocytes and macrophages.

These problems, and others, associated with the delivery of therapeutic pseudo-peptides to cells are successfully addressed by the present invention.

Conventional means for delivering bioactive agents are often limited by biological, chemical, and physical barriers. The barriers may arise from the nature of the delivery vehicle, the location of the target it is intended to reach, or from the target itself.

For example, a therapeutic agent may first encounter a physical barrier such as skin or an organ membrane before reaching its intended target. An agent may be hindered by chemical barriers, such as degrading enzymes, lipid bi-layers, and physiological variations in pH.

Such barriers are particularly significant in the oral delivery of drugs. Oral delivery of therapeutic agents is a desirable route of administration, generally because it facilitates greater levels of patient compliance. Thus, a greater number of the patient population achieves maximum benefit from the therapy. Yet oral administration of drugs is often hindered by physiological and biochemical barriers before the active agent reaches its target. For example, an active agent must overcome varying pH levels in the gastrointestinal tract, powerful digestive enzymes, and relatively impermeable gastrointestinal membranes. Numerous pharmacological agents are not typically amenable to oral administration, including biologically active polypeptides and proteins, such as calcitonin and insulin, polysaccharides, and other organic agents. The bioactive peptides in particular are rapidly rendered ineffective or destroyed by acid hydrolysis in the stomach or lower gastrointestinal tract and by enzymes capable of cleaving peptide bonds.

Earlier methods for orally administering vulnerable pharmacological agents have relied on the co-administration of adjuvants (e.g., resorcinols and non-ionic surfactants such as polyoxyethylene oleyl ether and n-hexadecylpolyethylene ether) to increase artificially the permeability of the intestinal walls, as well as the co-administration of enzymatic inhibitors (e.g. pancreatic trypsin inhibitors, diisopropylfluorophosphate (DFF) and trasylol) to inhibit enzymatic degradation.

Liposomes have also been described as drug delivery systems for insulin and heparin. See, for example, U.S. Pat. No. 4,239,754; Patel et al. (1976), FEBS Letters, 62, p. 60; and Hashimoto et al. (1979), Endocrinology Japan, 26, p. 337, the disclosures of which are herein incorporated specifically by reference.

However, broad spectrum use of such drug delivery systems is precluded because: (1) the systems require toxic amounts of adjuvants or inhibitors; (2) suitable low molecular weight cargos, i.e. active agents, are not available; (3) the systems exhibit poor stability and inadequate shelf life; (4) the systems are difficult to manufacture; (5) the systems fail to protect the active agent; (6) the systems adversely alter the active agent; or (7) the systems fail to allow or promote absorption of the active agent.

More recently, synthetic amino acid polymers or proteinoids, forming microspheres, have been described for encapsulating pharmaceuticals. For example, U.S. Pat. No. 4,925,673 (the '673 patent), the disclosure which is hereby incorporated specifically by reference, describes such microsphere constructs, as well as methods for their preparation and use. The '673 patent also describes microspheres which encapsulate pharmaceutical agents for delivery into the gastrointestinal tract or into the blood.

While the proteinoid microspheres described in the '673 patent are useful for their intended purposes, the physicochemical properties of the proteinoid microspheres, such as light sensitivity, shelf life and the selectivity of their solubility in various portions of the gastrointestinal tract, could be significantly improved. Additionally, there is a need in the art for delivery vehicles that can facilitate the delivery of a broader range of active agents such as polar drugs.

There is still a need for simple, inexpensive delivery systems which are easily prepared and which can deliver bioactive peptide agents and modified versions thereof to their intended anatomical sites in vivo.

Definitions:

pseudo-peptide: a chemical modification of one or several amino acid residues constituting the peptide or of their bonds such as, but not limited to, use of amino acids in their D-configuration; use of N-methyl amino acids; replacing one or more peptidic bonds (CO—NH) by a reduced bond (CH2-NH), and/or by NH—CO, CH2-CH2, CO—CH2, CHOH—CH2, or CH2-);

lipopeptides: a combination of natural peptides (not involving any modified amino acids or modified bonds) and a lipid moiety;

lipopseudo-peptides: pseudo-peptides coupled with a lipid moiety.

chemically modified amino acid aa: an amino acid sequence wherein at least one of the amino acid residues in their bonds is modified as set forth above in these definitions.

### SUMMARY OF THE INVENTION

The pseudo-peptide derivatives of the present invention provide a prodrug for the treatment of physiological conditions comprising a carrier moiety selected from the group cinnamoyl, benzoyl, phenylacetyl, 3,4-methylenedioxycinnamoyl, and 3,4,5-trimethoxycinnamoyl. Preferably, the carrier moiety is chemically linked to a therapeutic pseudo-polypeptide containing a chemically modified amino acid, aaₙ. Preferably, n is an integer from 2 to 40, and the pseduo-polypeptide is a poorly absorbed compound. More preferably, the pseudo-polypeptide has the formula aaₙ, wherein n is an integer from 20 to 40. Still even more preferably, n is 30.

In another variation, the prodrug of the present invention comprises a non-therapeutic linker species linking the polypeptide to the carrier moiety.

Preferably, the non-therapeutic linker species is an amino acid.

In another embodiment, the present invention contemplates a pharmaceutical composition comprising a carrier moiety selected from the group consisting essentially of cinnamoyl, benzoyl, phenylacetyl, 3,4 methylenedioxycinnamoyl and 3,4,5-trimethoxycinnamoyl chemically linked to a therapeutic pseudo-polypeptide having the formula aan, where aa is a chemically modified amino acid or a chemical or structural variation thereof, where n is an integer from 2 to 40, wherein the pseudo-polypeptide is poorly absorbed orally, and a pharmaceutically acceptable carrier.

In yet another embodiment, the present invention provides a method for enhancing the oral bioavailability of therapeutic pseudo-polypeptides having the formula formula aaₙ, where aa is a chemically modified amino acid or a chemical or structural variation thereof. Preferably, n is an integer from 2 to 40, and the pseudo-polypeptide is poorly absorbed orally. The polypeptide is chemically linked to a carrier moiety selected from the group consisting essentially of cinnamoyl, benzoyl, phenylacetyl, 3,4-methylenedioxycinnamoyl and 3,4,5-trimethoxycinnamoyl to form a prodrug.

Preferably, the pseudo-polypeptide is chemcially linked to the carrier through a non-therapeutic linker species.

More specifically, the linker species is an amino acid.

In yet another embodiment, the present invention provides a method for the treatment of a physiological condition through the oral administration of a therapeutically effective pseudo-polypeptide comprising the steps of:

(a) chemically linking a therapeutic pseudo-polypeptide having the formula aaₙ to a carrier moiety selected from the group consisting essentially of cinnamoyl, benzoyl, phenylacetyl, 3,4-methylenedioxycinnamoyl and 3,4,5-trimethoxycinnamoyl to form a prodrug. Specifically, aa is a chemically modified amino acid or a chemical or structural variation thereof, and n is an integer from 2 to 40, and the pseudo-polypeptide is poorly absorbed orally; and

(b) orally administering the prodrug to a patient exhibiting the physiological condition.

Preferably, the pseudo-polypeptide is chemically linked to the carrier moiety through a non-therapeutic linker species. More specifically, the linker species is an amino acid.

Yet another embodiment is a method for the controlled release of a therapeutically effective pseudo-polypeptide having the formula aaₙ. Specifically, aa is a chemically modified amino acid or a chemical or structural variation thereof, and n is an integer of from 2 to 40. Generally, the pseudo-polypeptide is poorly absorbed orally. The method preferably comprises the steps of:

(a) chemically linking the pseudo-polypeptide to a carrier moiety selected from the group consisting essentially of cinnamoyl, benzoyl, phenylacetyl, 3,4-methylenedioxycinnamoyl and 3,4,5-trimethoxycinnamoyl to form a prodrug; and

(b) orally administering the prodrug to a patient.

Preferably, the polypeptide is chemically linked to the carrier through a non-therapeutic linker species. More preferably, the linker species is an amino acid.

In still another embodiment, the present invention provides a method for improving the immune response of a mammal against chronic and latent viral infections and malignant cells comprising the step of administration to the mammal a pharmaceutical composition. Preferably, the route is oral. More preferably, the pharmaceutical composition is in a solid oral dosage form. Alternatively, the route of administration can be via injection.

### DETAILED DESCRIPTION OF THE INVENTION

In one embodiment, the present invention provides a prodrug for use in the treatment of physiological conditions comprising a carrier moiety selected from the group comprising cinnamoyl, benzoyl, phenylacetyl, 3,4-methylenedioxycinnamoyl and 3,4,5-trimethoxycinnamoyl, wherein the carrier moiety is chemically linked to a therapeutic pseudo-polypeptide of the formula aaₙ, where aa is an amino acid, or a chemical or structural variation thereof, where n is an integer from 2 to 40, and wherein the polypeptide is poorly absorbed orally. Preferably, in the prodrug of the invention, n is an integer from 20 to 40. More preferably, n is 30.

In an alternative variation, the prodrug of the present invention further comprises a non-therapeutic linker species linking the pseudo-polypeptide to the carrier moiety. Preferably, the linker species is an amino acid. Thus, the prodrug of the present invention can be viewed as a three-component entity: the first, therapeutically active component is the pseudo-polypeptide; the second is the linker species, possibly an additional, non-therapeutic amino acid; and the third is the carrier moiety.

When delivered orally, the prodrug of the present invention is capable of delivery of a systemic dose of the active drug species to a patient ingesting the prodrug. The active pseudo-polypeptide, in unmodified form, is normally degraded in the gastrointestinal tract to non-therapeutic forms. However, when formulated as the prodrug of the present invention, it survives and is processed in vivo, most likely by enzymatic hydrolysis in the liver, to release the active form at a controlled, bioavailable rate. An added benefit of the present invention is that the kinetics of such breakdown to release the active ingredient are significantly slower than that associated with other methods of delivery of the unmodified pseudo-polypeptide, effectively permitting a controlled release of the active species into the patient's system.

In another embodiment, the present invention contemplates a pharmaceutical composition comprising a carrier moiety selected from the group comprising cinnamoyl, benzoyl, phenylacetyl, 3,4-methylenedioxycinnamoyl and 3,4,5-trimethoxycinnamoyl chemically linked to a therapeutic pseudo-polypeptide of the general formula aaₙ, where aa is a chemically modified amino acid, or a chemical or structural variation thereof, where n is an integer of from 2 to 40, wherein the pseudo-polypeptide is poorly absorbed orally, and a pharmaceutically effective adjuvant species.

As would be recognized by one of skill in the appropriate art area, only one or more of the amino acids of the therapeutically active polypeptides used in conjunction with the present invention need be modified chemically or conformationally without significantly diminishing the pharmacological activity of the therapeutic entity.

The prodrugs of the present invention are formulated into pharmaceutical compositions that contain an efficacious amount of at least one lipopseudo-peptide in combination with an inert pharmaceutical vehicle.

The pharmaceutical compositions contain the derivatives alone or in combination with other medications.

The pharmaceutical compositions of the invention can be administered in different forms and by different routes, namely nasal, rectal and oral and by infection.

In the case of administration by the oral route, they may be used in the form of tablets, pills, lozenges, gelatin capsules and even liposomes. These compositions advantageously contain from 0.05 μg to 100 mg of lipo-pseudo peptide, per dosage unit.

The pseudo-peptides of the invention are particularly useful in improving the immune response against agents such as viruses for which antibodies have been shown to enhance infectivity, particularly to provide such a response against Goth chronic and latent viral infectious and malignant cells.

The present invention also provides a method for enhancing the oral availability of therapeutic pseudo-polypeptides of the formula formula aaₙ, where aa is a chemically modified amino acid, or a chemical or structural variation thereof, where n is an integer of from 2 to 40, and wherein the pseudo-polypeptide is poorly absorbed orally, wherein the method comprises the step of chemically linking the pseudo-polypeptide to a carrier moiety selected from the group including cinnamoyl, benzoyl, phenylacetyl, 3,4-methylenedioxycinnamoyl and 3,4,5-trimethoxycinnamoyl to form a prodrug. Preferably, this embodiment of the present invention provides a prodrug where the pseudo-polypeptide is chemically linked to the carrier moiety through a non-therapeutic linker species. More preferably, the linker species is an amino acid.

The instant invention also encompasses a method for the treatment of a physiological condition through the oral administration of a therapeutically effective species comprising the steps of chemically linking a therapeutic pseudo-polypeptide of the formula aaₙ, where aa is a chemically modified amino acid, or a chemical or structural variation thereof, where n is an integer from 2 to 40, and wherein the pseudo-polypeptide is poorly absorbed orally, to a carrier moiety selected from the group including cinnamoyl, benzoyl, phenylacetyl, 3,4-methylenedioxycinnamoyl and 3,4,5-trimethoxycinnamoyl to form a prodrug, and orally administering the prodrug to a patient exhibiting the physiological condition. Preferably, in the practice of the method of the present invention, the polypeptide is chemically linked to the carrier moiety through a non-therapeutic linker species. More preferably still, the linker species is an amino acid.

Thus, utilizing the present invention, it is possible to treat physiological conditions through oral administration of therapeutically active pseudo-polypeptides that would normally have to be administered through considerably less desirable routes of administration, such as by injection.

In still another embodiment, the invention of the instant application provides for a method for the controlled release administration of a therapeutically effective pseudo-polypeptide of the formula aaₙ, where aa is a chemically modified amino acid, or a chemical or structural variation thereof, where n is an integer from 2 to 40, and wherein the pseudo-polypeptide is poorly absorbed orally, comprising the steps of chemically linking the polypeptide to a carrier moiety selected from the group comprising cinnamoyl, benzoyl, phenylacetyl, 3,4-methylenedioxycinnamoyl and 3,4,5-trimethoxycinnamoyl to form a prodrug, and orally administering the prodrug to a patient. In a preferred embodiment, the pseudo-polypeptide is chemically linked to the carrier moiety through a non-therapeutic linker species, and, more preferably still, the linker species is an amino acid. Due to the kinetics of the hepatic degradation of the prodrug of the present invention, the therapeutically active polypeptide species is released to the patient's system over relatively long periods of time, dosage dependent, to a maximum of nearly twenty-four hours.

### EXAMPLE 1

A series of lipo-pseudo-peptides was synthesized and then evaluated to determine their ability to induce T-cell proliferation in a skin immunization model.

Model: Nine-week-old mice were immunized by application on bare skin of 100 μg of N_{ef(66-97)} peptide sequence and of its modifications in addition to 5 μg of choleric toxin, and 100 μg of oligodeoxynucleotide containing a CpG moiety (Immunology, (2002), 104:1-14); 2 weeks later splenocytes were collected and grown 4 days in the presence of 4 difference concentrations of N_{ef(66-97)} peptide. The proliferation was measured by incorporating tritiated thymidine.

Formulations:

Nef=Nef₍₆₆₋₉₇₎

Lipo=Nef₍₆₆₋₉₇₎-palmitoyl lysilamide

C0=Cinnamoyl-Nef₍₆₆₋₉₇₎G₉₅ψ(CH₂—CH₂)G₉₆

CC5=Cinnamoyl-aminovaleryl-Nef₍₆₆₋₉₇₎G₉₅ψ(CH₂—CH₂)G₉₆

CC8=Cinnamoyl-aminooctanoyl-Nef₍₆₆₋₉₇₎G₉₅ψ(CH₂—CH₂)G₉₆

Pivgal=D-Gal(OPiv)₄-hydroxyvaleryl-aminooctanoyl-Nef₍₆₆₋₉₇₎G₉₅ψ(CH₂—CH₂)G₉₆

where G₉₅ψ(CH₂—CH₂)G₉₆ represents the pseudo-peptidic chemical modification of G₉₅-G₉₆ of the Ne_{ef(66-97)} sequence.

TABLE 1
Proliferation Index

| Conc. of Nef peptide: | Nef | Lipo | C0 | CC5 | CC8 | Pivgal |
|---|---|---|---|---|---|---|
| 50 | 10 | 07 | 12 | 20 | 16 | 10 |
| 5 | 13 | 08 | 14 | 21 | 20 | 08 |
| 0.5 | 10 | 06 | 12 | 22 | 22 | 05 |
| .05 | 03 | 03 | 08 | 11 | 10 | 02 |

The results shown in Table 1 demonstrate that CC5 and CC8 have the best proliferation index. Standard lipopeptides are equivalent to baseline (Nef) as well as CO and Pivgal. The addition of a pseudo-peptidic modification coupled with the covalent binding of a bulky lipid moiety (Cinnamoyl+fatty acid in C5 or C8) is required to enhance the activity compared to the baseline. Lipopeptides with C16 fatty acid (hexadecanoic acid derivative) without the pseudo-peptide modification are not as effective as the lipo-pseudo-peptides.

What is claimed is:

1. A prodrug for use in the treatment of physiological conditions comprising a carrier moiety selected from the group consisting essentially of cinnamoyl, benzoyl, phenylacetyl, 3,4-methylenedioxycinnamoyl and 3,4,5-trimethoxycinnamoyl, wherein the carrier moiety is chemically linked to a therapeutic pseudo-polypeptide having formula aaₙ, where aa is a chemically modified amino acid or a chemical or structural variation thereof, where n is an integer from 2 to 40, and wherein the pseudo-polypeptide is poorly absorbed orally.

2. The prodrug of claim 1, wherein n is an integer from 20 to 40.

3. The prodrug of claim 1, wherein n is 30.

4. The prodrug of claim 1, wherein the prodrug further comprises a non-therapeutic linker species linking the polypeptide to the carrier moiety.

5. The prodrug of claim 4, wherein the non-therapeutic linker species is an amino acid.

6. A pharmaceutical composition comprising a carrier moiety selected from the group consisting essentially of cinnamoyl, benzoyl, phenylacetyl, 3,4 methylenedioxycinnamoyl and 3,4,5-trimethoxycinnamoyl chemically linked to a therapeutic pseudo-polypeptide having the formula aaₙ, where aa is a chemically modified amino acid or a chemical or structural variation thereof, where n is an integer from 2 to 40, wherein the pseudo-polypeptide is poorly absorbed orally, and a pharmaceutically acceptable carrier.

7. A method for enhancing the oral availability of therapeutic pseudo-polypeptides having the formula formula aaₙ, where aa is a chemically modified amino acid or a chemical or structural variation thereof, where n is an integer from 2 to 40, and wherein the pseudo-polypeptide is poorly absorbed orally, comprising the step of chemically linking the polypeptide to a carrier moiety selected from the group consisting essentially of cinnamoyl, benzoyl, phenylacetyl, 3,4-methylenedioxycinnamoyl and 3,4,5-trimethoxycinnamoyl to form a prodrug.

8. The method of claim 7, wherein the pseudo-polypeptide is chemically linked to the carrier moiety through a non-therapeutic linker species.

9. The method of claim 8, wherein the linker species is an amino acid.

10. A method for the treatment of a physiological condition through the oral administration of a therapeutically effective pseudo-polypeptide comprising the steps of:
(a) chemically linking a therapeutic pseudo-polypeptide having the formula aaₙ, where aa is a chemically modified amino acid or a chemical or structural variation thereof, where n is an integer from 2 to 40, and wherein the pseudo-polypeptide is poorly absorbed orally, to a carrier moiety selected from the group consisting essentially of cinnamoyl, benzoyl, phenylacetyl, 3,4-methylenedioxycinnamoyl and 3,4,5-trimethoxycinnamoyl to form a prodrug; and
(b) orally administering the prodrug to a patient exhibiting the physiological condition.

11. The method of claim 10, wherein the pseudo-polypeptide is chemically linked to the carrier moiety through a non-therapeutic linker species.

12. The method of claim 11, wherein the linker species is an amino acid.

13. A method for the controlled release administration of a therapeutically effective pseudo-polypeptide having the formula aaₙ, where aa is a chemically modified amino acid or a chemical or structural variation thereof, where n is an integer of from 2 to 40, and wherein the pseudo-polypeptide is poorly absorbed orally, comprising the steps of:
(a) chemically linking the pseudo-polypeptide to a carrier moiety selected from the group consisting essentially of cinnamoyl, benzoyl, phenylacetyl, 3,4-methylenedioxycinnamoyl and 3,4,5-trimethoxycinnamoyl to form a prodrug; and
(b) orally administering the prodrug to a patient.

14. The method of claim 13, wherein the polypeptide is chemically linked to the carrier moiety through a non-therapeutic linker species.

15. The method of claim 14, wherein the linker species is an amino acid.

16. A method for improving the immune response of a mammal against chronic and latent viral infections and malignant cells comprising the step of administration to the mammal a pharmaceutical composition according to claim 6.

17. The method of claim 16, wherein the route of administration is oral.

18. The method of claim 17, wherein the oral route of administration comprises administering the pharmaceutical composition in a solid oral dosage form.

19. The method of claim 16, wherein the route of administration is via injection.
